(19) United States
(12) Patent Application Publication
(10) Pub. No.: US 2009/0214563 A1
(43) Pub. Date: Aug. 27, 2009

(54) TREATMENT FOR ANTIPHOSPHOLIPID-SYNDROME-RELATED PREGNANCY COMPLICATIONS

(75) Inventors: Daniel Kirchhofer, Los Altos, CA (US); Jane E. Salmon, New York, NY (US); Guillermina Girardi, New York, NY (US)

Correspondence Address: MORRISON & FOERSTER LLP, 755 PAGE MILL RD, PALO ALTO, CA, 94304-1018, US

(73) Assignee: Genentech, Inc.

(21) Appl. No.: 12/090,852

(22) PCT Filed: Oct. 20, 2006
(86) PCT No.: PCT/US2006/060103

§ 371 Date: Oct. 29, 2008

Related U.S. Application Data
(60) Provisional application No. 60/728,831, filed on Oct. 21, 2005.

(51) Int. Cl. A61K 39/395; A61K 38/02; A61K 31/727

(52) U.S. Cl. 424/145.1; 424/158.1; 514/2; 514/56

(57) ABSTRACT

The present application provides compositions and methods for treating antiphospholipid-syndrome-related pregnancy complications with tissue factor antagonists.

### FIELD OF THE INVENTION

The present invention relates to methods for treating antiphospholipid-syndrome-related pregnancy complications.

### BACKGROUND OF THE INVENTION

The antiphospholipid syndrome (APS) is characterized by the presence of antiphospholipid (aPL) antibodies, which are associated with thrombosis. APS also is a leading cause of miscarriage and maternal and fetal morbidity. In women with APS, pregnancies that proceed into the third trimester have high incidence of preeclampsia and subsequent intrauterine fetal growth restriction, abruption placentae, and premature birth (Lima et al., Clin. Exp. Rheumatol. 14:131-36 (1996)). In vivo and in vitro studies have demonstrated that aPL antibodies trigger activation of endothelial cells, monocytes, neutrophils, and platelets, and produce inflammation, thrombosis, and tissue damage.

Several hypotheses have been proposed to explain the mechanism by which aPL antibodies promote thrombosis, including roles for both procoagulant and anticoagulant effects (Levine et al., N. Engl. J. Med. 346:752-63 (2002)). A first theory implicates activation of endothelial cells, a second focuses on oxidant-mediated injury to the vascular endothelium, and a third proposes that aPL antibodies affect the function of proteins involved in regulation of coagulation (Levine et al., supra). Although a role for tissue factor has been suggested in pathogenesis of aPL-antibody-related thrombosis, it remains unclear which of these mechanisms are responsible for its various pathologies (Amengual et al., Thromb. Haemost. 79: 276-81 (1998)). However, previous studies of proteins involved in coagulation concluded that placental coagulant pathways involving tissue factor, thrombomodulin, and annexin V do not contribute to APS-related pregnancy complications (Lakasing et al., Am. J. Obstet. Gynecol. 181:180-89 (1999)).

The anticoagulant heparin is the standard treatment used to prevent obstetric complications in pregnant women with APS. However, heparin treatment is not fully effective and has some risks (Ahmed et al., Am. J. Med. Sci. 324 (5):279-80 (2002)). Accordingly, there is a need for additional compositions and methods for treating APS-associated pregnancy complications.

### SUMMARY OF THE INVENTION

The present invention is based in part on the surprising discovery that treatment with tissue factor antagonists blocks APS-induced pregnancy complications, including fetal loss and growth restriction.

In some embodiments, the invention provides a method of treating a patient at risk of APS-related pregnancy complications comprising administering to said patient a tissue factor antagonist. In some embodiments, the tissue factor antagonist antagonizes a tissue factor activity selected from the group consisting of: binding to factor VII, binding to factor VIIa, effectuating the proteolysis of factor VII, effectuating the proteolysis of factor IX, and effectuating the proteolysis of factor X. In some embodiments, the tissue factor antagonist is an antibody, including a monoclonal antibody. In some embodiments, the tissue factor antagonist is a peptide. In some embodiments, the tissue factor antagonist is administered in the second trimester of pregnancy. In some embodiments, the method further comprises administering to the patient an anticoagulant, including heparin.

In some embodiments, the invention provides a kit comprising: (a) a tissue factor antagonist; (b) a container containing said tissue factor antagonist; and (c) a label affixed to said container, or a package insert included in said container, referring to the use of said antagonist in the treatment of APS-related pregnancy complications. In some embodiments, the tissue factor antagonist present in the kit is an antibody, including a monoclonal antibody. In some embodiments, the tissue factor antagonist is a peptide. In some embodiments, the kit further comprises an anticoagulant, including heparin.

### DETAILED DESCRIPTION OF THE PREFERRED EMBODIMENTS

Tissue factor (TF) is an integral membrane glycoprotein, which is the cell surface-expressed cofactor for the serine protease factor VIIa (FVIIa). TF triggers blood coagulation by combining with FVIIa to activate substrats factors VII IX, and X. The ensuing coagulation reactions result in the formation of a polymerized fibrin meshwork and platelet aggregates, which together form a hemostatic plug. The activity of TF/FVIIa has been implicated in numerous diseases, including cardiovascular diseases, cancer growth and metastasis, tumor angiogenesis and in inflammatory diseases, such as sepsis, rheumatoid arthritis and sickle cell anemia. General increased expression of TF by endothelial cells or monocytes has been observed in patients with APS (Amengual et al., supra), but no difference in expression was seen in placentas from women with APS as compared to controls (Lakasing et al., supra).

As used herein, the term “antiphospholipid syndrome” or “APS” refers to a clinical association between antiphospholipid antibodies and a syndrome of hypercoagulability (Levine et al., N. Eng. J. Med. 346:752-63 (2002)).

As used herein, the term “antiphospholipid-syndrome-related pregnancy complications” or “APS-related pregnancy complications” refers to increased fetal resorption, decreased fetal weight and/or increased miscarriage frequency in a female mammal with antiphospholipid syndrome. In humans, the criteria for classifying a patient as having APS-related pregnancy complications include the presence of aPL antibodies and: (1) one or more unexplained deaths of morphologically normal fetuses at or after the 10th week of gestation; or (2) one or more premature births of morphologically normal neonates at or before the 34th week of gestation; or (3) three or more unexplained consecutive spontaneous abortions or miscarriages before the 10th week of gestation (Levine et al., supra).

As used herein, the terms “TF,” “tissue factor,” “tissue factor protein” and “mammalian tissue factor protein” refer to a polypeptide having an amino acid sequence corresponding to a naturally occurring mammalian tissue factor (e.g., U.S. Pat. No. 6,274,142; Fisher et al. Thromb. Res. 48:89-99 (1987); Morrissey et al., Cell 50:129-35 (1987)). TF occurs naturally in humans as well as other animal species such as rabbits, rats, pigs, non-human primates, horses, mice, and sheep (see, e.g., Hartzell et al., Mol. Cell. Biol. 9:2567-73 (1989); Andrews et al., Gene 98:265-69 (1991); and Takayenik et al., Biochem. Biophys. Res. Comm. 181:1145-50 (1991)). The amino acid sequences of mammalian tissue factor proteins are generally known or obtainable through conventional techniques.

As used herein, the terms “TF antagonist” and “tissue factor antagonist” refers to a substance which inhibits or neutralizes an activity of TF. Such antagonists may accomplish this effect in various ways and need not act directly on TF. First, one class of TF antagonists binds to tissue factor protein with sufficient affinity and specificity to inhibit its ability to bind to factor VII or VIIa or effect the proteolysis of factors VII, IX, or X when in complex with factor VII or VIIa. Included within this group of molecules are certain antibodies and antibody fragments (such as, for example, F(ab) or F(ab′)₂ molecules). Another class of TF antagonists antagonizes TF activity by creating a complex of molecules, e.g., the naturally occurring tissue factor pathway inhibitor-1 (TFPI-1) which comprises lipoprotein associated coagulation inhibitor which forms an inactive complex of TF, factor VII, factor X and phospholipid (see, e.g., Broze et al., Proc. Natl. Acad. Sci. USA 84: 1886-90 (1987)). Another class of TF antagonists are fragments of TF protein, fragments of factor VII or small organic molecules, i.e. peptides or peptidomimetics, that bind to TF, thereby inhibiting the formation of the TF-factor VII complex or inhibit the activation of factors IX and X by TF and FVII/FVIIa (see, e.g., WO 01/01749, WO 01/10892, and US Patent Publication 2001/0048924). Yet another class of TF antagonists will inactivate TF protein or the tissue factor/factor VIIa complex by cleavage, e.g. a specific protease. A fifth class of TF antagonists block the binding of TF protein to factor VII, e.g., a factor VII antibody directed against a domain of factor VII which is involved in TF binding.

As used herein, the term “patient” for purposes of the treatment of, alleviating the symptoms of, or diagnosis of APS-related pregnancy complications refers to any mammal, including humans, domestic and farm animals, and zoo, sports, or pet animals, such as dogs, cats, cattle, horses, sheep, pigs, goats, rabbits, chimps, baboons, monkeys, etc. Preferably, the patient is human.

As used herein, the term “anticoagulant” refers to a substance that can prevent, inhibit or prolong blood coagulation in an in vitro or in vivo assay of blood coagulation. Anticoagulants include, e.g., heparin and aspirin. Blood coagulation assays are known in the art and include, for example, prothrombin time assays, the human ex vivo thrombosis model described by Kirchhofer et al., Arterioscler. Thromb. Vasc. Biol. 15: 1098-1106 (1995); and Kirchhofer et al., J. Clin. Invest. 93: 2073-83 (1994), and assays based on the measurement of Factor X activation in human plasma.

An antibody to tissue factor protein is used in some embodiments of the invention. As used herein, the term “anti-tissue factor antibody” or “ATF Ab” refers to an antibody that specifically binds to a tissue factor. In some embodiments, the ATF Ab is an antibody described in PCT publication WO 01/70984.

The term “antibody” (Ab) is used herein in the broadest sense and specifically covers, for example, single monoclonal antibodies, multispecific antibodies (such as bispecific antibodies), antibody compositions with polyepitopic specificity, polyclonal antibodies, single chain antibodies, and fragments of antibodies (see below) as long as they specifically bind a native polypeptide and/or exhibit a biological activity or immunological activity of this invention.

As used herein, an “intact” antibody is one which comprises an antigen-binding site as well as a CL and at least heavy chain constant domains CH1, CH2 and CH3. The constant domains can be native sequence constant domains (e.g., human native sequence constant domains) or amino acid sequence variant thereof. Preferably, the intact antibody has one or more effector functions.

As used herein, a “species-dependent antibody” is an antibody which has a stronger binding affinity for an antigen from a first mammalian species than it has for a homologue of that antigen from a second mammalian species. Normally, the species-dependent antibody “binds specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50-fold, or at least about 500-fold, or at least about 1000-fold, weaker than its binding affinity for the human antigen. The species-dependent antibody can be of any of the various types of antibodies as defined above, but preferably is a humanized or human antibody.

As used herein, the term “monoclonal antibody” refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they can be synthesized uncontaminated by other antibodies. The modifier “monoclonal” is not to be construed as requiring production of the antibody by any particular method. For example, the monoclonal antibodies useful in the present invention can be prepared by the hybridoma methodology first described by Kohler et al., Nature 256: 495 (1975), or can be made using recombinant DNA methods in bacterial, eukaryotic animal or plant cells (see, e.g., U.S. Pat. No. 4,816,567). Anti-TF monoclonal antibodies have been prepared (see, e.g., Carson et al., Blood 66 (1): 152-56 (1985)). The “monoclonal antibodies” also can be isolated from phage antibody libraries using the techniques described in Clackson et al., Nature 352: 624-28 (1991), Marks et al., J. Mol. Biol. 222: 581-97 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit TF antagonist activity (see U.S. Pat. No. 4,816,567; and Morrison et al., Proc. Natl. Acad. Sci. USA 81: 6851-55 (1984)). Chimeric antibodies of interest herein include “primatized” antibodies comprising variable domain antigen-binding sequences derived from a non-human primate (e.g., Old World monkey, ape, etc.) and human constant region sequences.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis described in Munson et al., Anal. Biochem. 107: 220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal, e.g., by i.p. injection of the cells into mice.

The monoclonal antibodies secreted by the subclones are suitably separated from the culture medium, ascites fluid, or serum by conventional antibody purification procedures such as, e.g., affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as one source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opin. in Immunol. 5: 256-62 (1993) and Plückthun, Immunol. Revs. 130: 151-88 (1992).

Monoclonal antibodies or antibody fragments can also be isolated from antibody phage libraries generated using the techniques described in McCafferty et al., Nature 348: 552-554 (1990). Clackson et al., Nature 352: 624-28 (1991) and Marks et al., J. Mol. Biol. 222: 581-97 (1991) describe the isolation of murine and human antibodies, respectively, using phage libraries. Subsequent publications describe the production of high affinity (nM range) human antibodies by chain shuffling (Marks et al., Bio/Technology 10: 779-83 (1992)), as well as combinatorial infection and in vivo recombination as a strategy for constructing very large phage libraries (Waterhouse et al., Nucl. Acids. Res. 21: 2265-66 (1993)). Thus, these techniques are viable alternatives to traditional monoclonal antibody hybridoma techniques for isolation of monoclonal antibodies.

The DNA that encodes the antibody may be modified, for example, by substituting human heavy chain and light chain constant domain (CH and CL) sequences for the homologous murine sequences (U.S. Pat. No. 4,816,567; and Morrison, et al., Proc. Natl Acad. Sci. USA 81:6851 (1984)), or by fusing the immunoglobulin coding sequence with all or part of the coding sequence for a non-immunoglobulin polypeptide. The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. “Humanized” forms of non-human (e.g. rodent) antibodies are chimeric antibodies that contain minimal sequences derived from non-human antibodies. Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can essentially be performed following the method of Winter and co-workers (Jones et al., Nature 321: 522-25 (1986); Reichmann et al., Nature 332: 323-27 (1988); Verhoeyen et al., Science 239: 1534-36 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151: 2296 (1993); Chothia et al., J. Mol. Biol. 196: 901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA 89: 4285 (1992); Presta et al., J. Immunol. 151: 2623 (1993)).

It is further important that antibodies be humanized with retention of high binding affinity for the antigen and other favorable biological properties. To achieve this goal, humanized antibodies may, e.g., be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

A fully humanized anti-TF antibody, D3H44, has been produced (Presta et al., Thromb. Haemost. 85, 379-79 (2001)). In agreement with the excellent potency of the murine D3 antibody from which it was derived (Kirchhofer et al., Thromb. Haemost. 84, 1072-81 (2000)), D3H44 is a potent anticoagulant both in vitro (Presta et al., (2001) supra) and in vivo (Bullens et al., Thromb. Haemost. (Suppl) Abstract #P1388 (2001)).

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germline mutant mice results in complete inhibition of endogenous antibody production. Transfer of the human germline immunoglobulin gene array into such germline mutant mice will result in the production of human antibodies upon antigen challenge. See, e.g., Jakobovits et al., Proc. Natl. Acad. Sci. USA 90: 2551 (1993); Jakobovits et al., Nature 362: 255-58 (1993); Bruggemann et al., Year in Immuno. 7:33 (1993); U.S. Pat. Nos. 5,545,806, 5,569,825, 5,591,669, 5,545,807; and WO 97/17852.

Alternatively, phage display technology (McCafferty et al., Nature 348: 552-53 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats, reviewed in, e.g., Johnson and Chiswell, Curr. Opin. Struct. Biol. 3: 564-71 (1993). Several sources of V-gene segments can be used for phage display. Clackson et al., Nature 352: 624-28 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222: 581-97 (1991), or Griffith et al., EMBO J. 12: 725-34 (1993). See, also, U.S. Pat. Nos. 5,565,332 and 5,573,905. As discussed above, human antibodies also may be generated by in vitro activated human B cells (see U.S. Pat. Nos. 5,567,610 and 5,229,275).

In certain circumstances there are advantages of using antibody fragments, rather than whole antibodies. For example, the smaller size of the fragments allows for rapid clearance, and may lead to improved access to certain tissues.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (see, e.g., U.S. Pat. No. 5,641,870, Example 2; Zapata et al., Protein Eng. 8 (10): 1057-62 (1995)); single-chain antibody molecules; and multispecific antibodies formed from antibody fragments.

The expression “linear antibodies” generally refers to the antibodies described in Zapata et al. (1995), supra. Briefly, these antibodies comprise a pair of tandem Fd segments (VH-CH1-VH-CH1) which, together with complementary light chain polypeptides, form a pair of antigen binding regions. Linear antibodies can be bispecific or monospecific.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells

“Fv” is the minimum antibody fragment which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-48 (1993).

Bispecific antibodies are antibodies that have binding specificities for at least two different epitopes. Exemplary bispecific antibodies may bind to two different epitopes of tissue factor. Other such antibodies may combine a tissue factor binding site with a binding site for another protein.

Methods for making bispecific antibodies are known in the art. Traditional production of full-length bispecific antibodies is based on the co-expression of two immunoglobulin heavy chain-light chain pairs, where the two chains have different specificities (Millstein et al., Nature 305: 537-39 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10: 3655-59 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, CH2, and CH3 regions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Meth. Enzymol. 121: 210 (1986).

According to another approach described in U.S. Pat. No. 5,731,168, the interface between a pair of antibody molecules can be engineered to maximize the percentage of heterodimers which are recovered from recombinant cell culture. In one embodiment, the interface comprises at least a part of the CH3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 0308936). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-25 (1992) describe the production of a fully humanized bispecific antibody F(ab′)2 molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol. 148 (5): 1547-53 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90: 6444-48 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol. 152: 5368 (1994).

Use of antibodies with more than two valencies are contemplated. For example, trispecific antibodies can be prepared. Tutt et al., J. Immunol. 147: 60 (1991)

Antibodies with altered glycosylation patterns are contemplated for use in the invention. Altered glycosylation patterns may include deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the antibodies and peptides used in this invention are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of an antibody or polypeptide used in the methods of this invention.

To increase the serum half life of the antibody, one may incorporate a salvage receptor binding epitope into the antibody (especially an antibody fragment) as described in U.S. Pat. No. 5,739,277, for example.

Other modifications of the antibody or peptides used in the invention are contemplated. For example, the antibody or polypeptide can be linked to one of a variety of nonproteinaceous polymers, e.g., polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The antibodies and polypeptides of this invention can also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody or polypeptide can be prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-88 (1982) via a disulfide interchange reaction.

### II. Treatment With TF Antagonists

The tissue factor antagonist and the optional additional therapeutic used in the present invention may be provided to the recipient in combination. Medicaments are considered to be provided “in combination” with one another if they are provided to the patient concurrently, or if the time between the administration of each medicament is such as to permit an overlap of biologic activity.

An amount of tissue factor antagonist capable of treating APS-associated pregnancy complications when provided to a patient is a “therapeutically effective” amount. The therapeutically effective amount of a tissue factor antagonist will usually be administered using an amount per kilogram of patient weight determined by the ordinarily skilled physician. In some embodiments, this dosage may be administered by continual intravenous infusion over a period of between 75-180 minutes at a dose of about in the range of 0.01-25.0 milligrams per kilogram of patient weight. As would be apparent to one of ordinary skill in the art, the required dosage of the tissue factor antagonist for use in the treatment of APS-associated and, optionally, any other therapeutic will depend upon the severity of the condition of the patient, and upon such criteria as the patient's height, weight, age, and medical history.

### III. Pharmaceutical Formulations

Therapeutic formulations of the antibodies, peptides and other molecules used in the present invention are prepared for storage by mixing those that have the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g. Zn-protein complexes); and/or non-ionic surfactants such as TWEEN™, PLURONICS™ or polyethylene glycol (PEG). The formulation preferably comprises the antibody, polypeptide or other molecule at a concentration of between 5-200 mg/ml, preferably between 10-100 mg/ml.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid.

The formulations to be used for in vivo administration must be sterile. This is readily accomplished by filtration through sterile filtration membranes.

### IV. Articles of Manufacture

In another embodiment of the invention, an article of manufacture containing materials useful for the treatment of APS-related pregnancy complications is provided. The article of manufacture comprises (1) a container, (2) a TF antagonist, and (3) a label or package insert comprising instructions on how to perform a method of this invention. Another embodiment of the invention is an article of manufacture containing materials useful for performing a method of the invention. Suitable containers include, for example, bottles, vials, syringes, etc. The container can be formed from a variety of materials such as glass or plastic. The container holds a composition which is effective for treating the condition and can have a sterile access port (for example the container may be an intravenous solution bag or a vial having a stopper pierceable by a hypodermic injection needle). The label or package insert indicates that the composition is used for treating APS-associated pregnancy complications. The label or package insert will further comprise instructions for administering a TF antagonist composition to the patient.

All publications (including patents and patent applications) cited herein are hereby incorporated by reference in their entirety.

Commercially available reagents referred to in the Examples were used according to manufacturer's instructions unless otherwise indicated. Unless otherwise noted, the present invention uses standard procedures of recombinant DNA technology, such as those described hereinabove and in the following textbooks: Sambrook et al., Molecular Cloning: A Laboratory Manual, New York: Cold Spring Harbor Press, 1989; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### 1. Tissue Factor (TF) Binds To Deciduas In A C5-Dependent Manner

### Antiphospholipid Antibody (aPL)

We obtained aPL-IgG from patients with APS (characterized by a high titer of aPL antibodies [>140 GPL units], thromboses, and/or pregnancy losses) and NH-IgG (normal human) from healthy individuals. The human monoclonal antibody aPL IgG1 (monoclonal antibody 519) has been previously described (Ikematsu et al., Arthritis Rheum. 41: 1026-39 (1998)). We purified IgG by affinity chromatography using Protein G Sepharose® chromatography columns (Amersham Pharmacia Biotech) and treated it to deplete endotoxin with Centriprep® ultracentrifugation devices (Millipore Corporation) and determined it to be free of endotoxin using the Limulus amebocyte lysate assay.

### Mouse Model For aPL-Induced Pregnancy Loss

A mouse model for aPL-induced pregnancy loss was used as described previously (Girardi et al., Nature Med. 10: 1222-26 (2004)). On day 8 of pregnancy, 6-8-week-old C5^{+/+} and C5^{-/-} females (Taconic Farms) were treated with intraperitoneal injections of aPL-IgG (10 mg), human aPL mAb (1 mg) or NH-IgG (10 mg). Mice were sacrificed 60 minutes after treatment and the deciduas were removed, frozen in O.C.T. compound and cut into 10 μm sections. We then stained the sections using anti-tissue-factor antibodies.

We observed intense TF staining of deciduas in C5^{+/+} mice treated with aPL-IgG. In contrast, C5^{-/-} mice showed no TF staining of deciduas.

### 2. Antiphospholipid Antibody-Induced Fetal Loss And Weight Restriction Is C5-Dependent

On days 8 and 12 of pregnancy, 6-8-week-old C5^{+/+} and C5^{-/-} females (Taconic Farms) were treated with intraperitoneal injections of aPL-IgG (10 mg), human aPL mAb (1 mg) or NH-IgG (10 mg). Mice were sacrificed on day 15 of pregnancy, fetuses weighed and fetal resorption frequencies calculated (number of resorptions/total number of formed fetuses and resorptions). Resorption sites are easily identified and result from loss of a previously viable fetus.

In C5^{+/+} mice we observed an antiphospholipid-antibody-dependent increase in fetal resorption frequency and a similar decrease in embryo weight. The mice treated with aPL-IgG had a 38±8% resorption frequency whereas mice treated with NH-IgG had a 10±7% resorption frequency (p<0.01). Average embryo weights were 221±31 mg with aPL-IgG treatment and 389±36 mg with NH-IgG treatment (p<0.01). In contrast, in C5^{-/-} we did not observe either effect. C5^{-/-} mice treated with aPL-IgG had a 11±3% fetal resorption frequency whereas mice treated with NH-IgG had a 10±4% resorption frequency. Average embryo weights were 371±32 mg with aPL-IgG treatment and 357±41 mg with NH-IgG treatment.

Two complement effector pathways are initiated by cleavage of C5: C5a, a potent anaphylatoxin and activator of endothelial cells and leukocytes, and C5b, which leads to formation of the C5b-9 membrane attack complex (MAC). In order to distinguish which of these is involved in the C5-dependent effects, we performed these experiments in C5aR^{-/-} mice and in C6^{-/-} mice. We observed that the outcome of pregnancies in C5aR^{-/-} mice was normal whereas C6-deficient mice showed no amelioration of aPL-IgG-induced pregnancy failure. In addition, we observed that C5aR^{-/-} mice showed no TF production whereas C6-deficient mice showed increased TF staining in decidual tissues. This indicated that C5 is involved in the C5-dependent effects and further suggested a role for TF in this process.

### 3. Treatment With An Anti-Tissue-Factor Antibody Blocks Antiphospholipid Antibody-Induced Fetal Loss And Weight Restriction

To directly show that TF is an essential mediator of aPL-antibody-induced fetal injury, we inhibited TF function with a monoclonal antibody against mouse TF (1H1; Kirchhofer et al., J. Thromb. Haemost. 3: 1098-99(2005)). Treatment with 1H1 (0.5 mg on days 6 and 10) prevented aPL-antibody-induced pregnancy losses and growth restriction. In mice treated with aPL-IgG alone we observed a 39±7% fetal resorption frequency as compared to an 11±3% frequency in mice treated with aPL-IgG and 1H1 (p<0.001). Average embryo weights were 234±37 mg with aPL-IgG treatment and 381±46 mg with aPL-IgG plus 1H1 treatment (p<0.005). These results confirmed that TF is a mediator of aPL-antibody-induced fetal injury and that C5a plays a critical role as a trigger of TF generation.

1. A method of treating a patient at risk of antiphospholipid-syndrome-related pregnancy complications comprising administering to said patient a tissue factor antagonist.

2. The method of claim 1, wherein said tissue factor antagonist antagonizes a tissue factor activity selected from the group consisting of: binding to factor VII, binding to factor VIIa, effectuating the proteolysis of factor VII, effectuating the proteolysis of factor IX, and effectuating the proteolysis of factor X.

3. The method of claim 1, wherein said tissue factor antagonist is an antibody.

4. The method of claim 3, wherein said antibody is a monoclonal antibody.

5. The method of claim 1, wherein said tissue factor antagonist is a peptide.

6. The method of claim 1, wherein said tissue factor antagonist is administered in the second trimester of pregnancy.

7. The method of any one of claims 1 to 6 further comprising administering to said patient an anticoagulant.

8. The method of claim 7, wherein said anticoagulant is heparin.

9. A kit comprising:
(a) a tissue factor antagonist;
(b) a container containing said tissue factor antagonist;
(c) a label affixed to said container, or a package insert included in said container, referring to the use of said antagonist in the treatment of antiphospholipid-syndrome-related pregnancy complications.

10. The kit of claim 9, wherein said tissue factor antagonist is an antibody.

11. The kit of claim 10, wherein said antibody is monoclonal.

12. The kit of claim 9, wherein said tissue factor antagonist is a peptide.

13. The kit of any one of claims 9-12 further comprising an anticoagulant.

14. The kit of claim 13, wherein said anticoagulant is heparin.

15. The method of claim 2, wherein said tissue factor antagonist is an antibody.

16. The method of claim 15, wherein said antibody is a monoclonal antibody.

17. The method of claim 2, wherein said tissue factor antagonist is a peptide.

18. The method of any one of claims 15-17 further comprising administering to said patient an anticoagulant.

19. The method of claim 18, wherein said anticoagulant is heparin.
